(19) United States
(12) Patent Application Publication
(10) Pub. No.: US 2021/0052914 A1
(43) Pub. Date: Feb. 25, 2021

(54) OPTICAL THERAPEUTIC APPARATUS AND OPTICAL THERAPEUTIC METHOD

(71) Applicant: OLYMPUS CORPORATION, Tokyo (JP)

(72) Inventor: Masahiro YOSHINO, Machida-shi, Tokyo (JP)

(73) Assignee: OLYMPUS CORPORATION, Tokyo (JP)

(21) Appl. No.: 17/092,403

(22) Filed: Nov. 9, 2020

Related U.S. Application Data
(63) Continuation of application No. PCT/JP2019/007365, filed on Feb. 26, 2019.

(30) Foreign Application Priority Data
May 15, 2018 (JP) 2018-093948

(51) Int. Cl. A61N 5/06; A61M 25/10

(52) U.S. Cl. CPC A61N 5/0603; A61N 2005/061; A61M 2210/1085; A61M 25/10

(57) ABSTRACT

An optical therapeutic apparatus includes: a liquid discharge catheter including a first tubular member; a balloon catheter including a shaft, a balloon and a second tubular member; and a light irradiation body. The balloon is positioned such that the balloon is exposed from a distal end of the liquid discharge catheter by bringing the second tubular member into contact with the first tubular member.

### CROSS REFERENCE TO RELATED APPLICATION

This application is a continuation application of PCT/JP2019/007365 filed on Feb. 26, 2019 and claims benefit of Japanese Application No. 2018-093948 filed in Japan on May 15, 2018, the entire contents of which are incorporated herein by this reference.

### BACKGROUND OF THE INVENTION

### 1. Field of the Invention

The present invention relates to an optical therapeutic apparatus for performing therapy by irradiating a portion to which therapy is applied with light, and an optical therapeutic method performed using the optical therapeutic apparatus.

### 2. Description of the Related Art

Recently, as a therapeutic method effective for cancer, photoimmuno therapy (PIT) and photodynamic therapy (PDT) are known.

Japanese Patent No. 6127045 discloses a method which kills cells. The method includes: a step of bringing cells which contain cell-surface proteins into contact with a therapeutically effective amount of one or a plurality of antibody-IR700 molecules, where the antibodies are specifically bonded to the cell-surface proteins; a step of irradiating the cells with a dose of at least 1 Jcm⁻² at a wavelength of 660 to 740 nm; and a step of bringing the cells into contact with one or a plurality of therapeutic agent after a lapse of approximately 0 to 8 hours from the irradiation of the cells thus killing the cells.

Tumors which are exemplified by cancer and the like which can be treated by the method include bladder cancer.

In performing therapy by irradiating a bladder cancer with light, it is necessary to introduce an optical treatment probe into a bladder, to inflate the bladder to a capacity optimum for treatment, and to irradiate the bladder with a large amount of light. Accordingly, the use of a balloon catheter is supposed in the therapeutic method.

To irradiate an entirety of a tissue having a large capacity such as a bladder with a required amount of light, an optical therapeutic probe to be inserted into the bladder includes a functional unit which diffuses light. Further, to prevent lowering of an amount of light by urine at the time of light irradiation, discharge of urine by a catheter is supposed.

### SUMMARY OF THE INVENTION

According to an aspect of the present invention, there is provided an optical therapeutic apparatus which includes: a liquid discharge catheter including a lumen for liquid discharge, and a first tubular member with which a proximal end of the lumen for liquid discharge communicates;

a balloon catheter including a second tubular member provided with a shaft which includes one end and another end and is insertable into and removable from the lumen for liquid discharge of the liquid discharge catheter, a balloon mounted on the one end of the shaft, and a port disposed on the other end of the shaft and through which a liquid for inflating the balloon can be injected, the balloon catheter including a lumen for injection which makes the port and the balloon communicate with the shaft; and

a light irradiation body configured to emit light having a preliminarily set wavelength from a diffuse functional unit of a probe distal end in a state where the light irradiation body is inserted into the lumen for injection mounted on the shaft of the balloon catheter, the probe distal end is positioned in the balloon, and the liquid is injected into the balloon, wherein

the balloon is positioned to be exposed from a distal end of the liquid discharge catheter by bringing the second tubular member into contact with the first tubular member.

According to another aspect of the present invention, there is provided an optical therapeutic method which includes: inserting a light irradiation body into a lumen for injection of a balloon catheter, the balloon catheter including a balloon on one end of a shaft, a port capable of injecting liquid for inflating the balloon on another end, and a lumen for injection communicating the port and the balloon with each other, and disposing a probe distal end of the light irradiation body in a vicinity of an opening for balloon of the lumen for injection;

introducing the shaft of the balloon catheter into a lumen for liquid discharge of the liquid discharge catheter, the lumen for liquid discharge being capable of discharging urine in a bladder;

disposing a distal end of the liquid discharge catheter in the bladder so as to dispose the balloon mounted on one end of the balloon catheter in the bladder;

removing the balloon catheter from the liquid discharge catheter and discharging the urine in the bladder;

inserting the balloon catheter into the lumen for liquid discharge and disposing the balloon in the bladder;

injecting the liquid into the balloon through the port of the balloon catheter and inflating the balloon by the liquid; and

emitting light having a preliminarily set wavelength from a light irradiation unit of the light irradiation body toward the bladder.

### BRIEF DESCRIPTION OF THE DRAWINGS

FIG. 1 is a view for describing an optical therapeutic apparatus;

FIG. 2A is a view for describing a step of disposing a distal end portion of a liquid discharge catheter in a bladder and a step of discharging urine;

FIG. 2B is a view for describing a step of inserting a balloon catheter into a lumen for liquid discharge of the liquid discharge catheter, the distal end portion of which is disposed in the bladder;

FIG. 2C is a view for describing a step of disposing a distal end portion of a shaft on which a balloon is mounted from a distal end surface of the liquid discharge catheter in the bladder;

FIG. 2D is a view for describing a step of inflating the bladder together with the balloon by injecting a liquid in a syringe into the balloon;

FIG. 2E is a view for describing a step of disposing a light irradiation unit of a fiber probe in a vicinity of an opening for balloon and irradiating the bladder with light;

FIG. 3A is a view for describing a probe-balloon integral set formed by integrally combining a probe for therapy and the balloon catheter;

FIG. 3B is a view for describing a step of inserting the probe-balloon integral set into the lumen for liquid discharge of the liquid discharge catheter, the distal end portion of which is disposed in the bladder;

FIG. 3C is a view for describing a step of inflating the bladder while inflating the balloon by injecting a liquid in a state where the balloon of the probe-balloon integral set is disposed in the bladder;

FIG. 4A is a view for describing another configurational example of a liquid discharge catheter;

FIG. 4B is a view for describing a probe-balloon-liquid discharge catheter integral set formed by integrally combining the liquid discharge catheter having another configuration and the probe-balloon integral set;

FIG. 4C is a view for describing a step of disposing a distal end portion of the liquid discharge catheter of the probe-balloon-liquid discharge catheter integral set in the bladder;

FIG. 4D is a view for describing a step of disposing the balloon in the bladder by withdrawing the liquid discharge catheter of the probe-balloon-liquid discharge catheter integral set from the bladder and a step of inflating the bladder while inflating the balloon;

FIG. 5A is a view showing an example of a case where the probe-balloon integral set is inserted into the lumen for liquid discharge of the liquid discharge catheter, and the distal end portion of the liquid discharge catheter is disposed in the bladder in a state where the intrusion of a liquid from a distal end portion of the liquid discharge catheter into the lumen for liquid discharge is blocked;

FIG. 5B is a view showing an example of a case where, after an arrangement state shown in FIG. 5A is taken, urine in the bladder is discharged by removing the prove-balloon integral set inserted into the lumen for liquid discharge of the liquid discharge catheter; and

FIG. 5C is a view showing an example of a case where, after a state shown in FIG. 5B is taken, the balloon is disposed in the bladder by inserting the probe-balloon integral set into the lumen for liquid discharge of the liquid discharge catheter again.

### DETAILED DESCRIPTION OF THE PREFERRED EMBODIMENT

Hereinafter, an embodiment of the present invention is described with reference to drawings.

In the respective drawings used in the description made hereinafter, for the sake of setting sizes of respective components substantially legible in the drawings, the scales of the respective components are made different from each other. In other words, the present invention is not limited to the number and the amounts of components, the shapes of the components, ratios between the sizes of the components, and the relative positional relationships among the components described in the drawings.

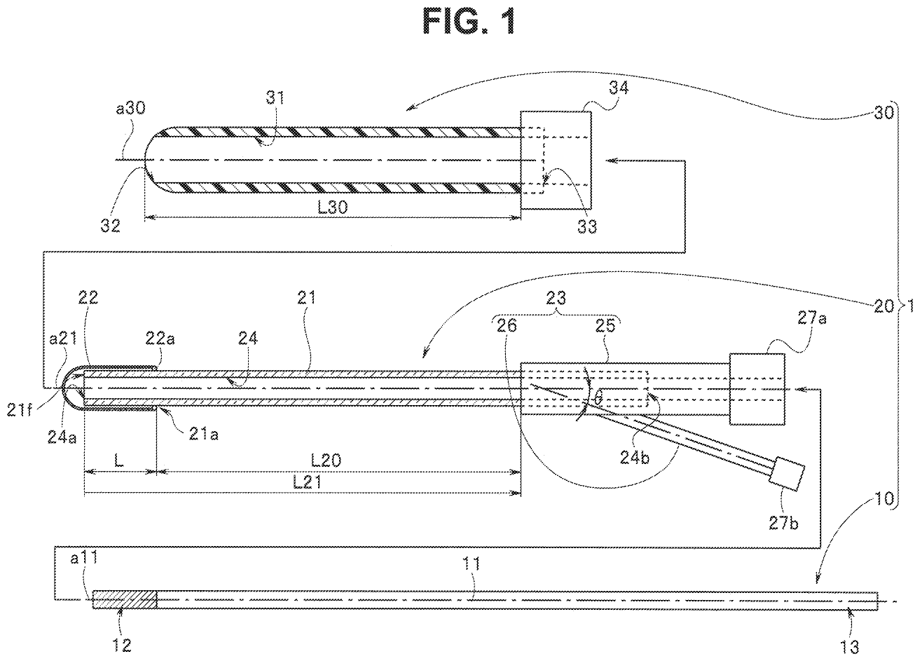

An optical therapeutic apparatus is described with reference to FIG. 1.

As shown in FIG. 1, the optical therapeutic apparatus 1 includes a probe 10 for therapy, a balloon catheter 20, and a liquid discharge catheter 30.

The probe 10 for therapy is a light irradiation body where a light irradiation unit 12 is mounted on an elongated fiber probe 11 having a preliminarily set outer diameter. The light irradiation unit 12 is a light diffusion functional unit, and is mounted on a side surface of a probe distal end which forms one end side of the fiber probe 11. A probe proximal end portion which forms the other end side of the fiber probe 11 forms a light source connection portion 13 connected to a light source device (not shown).

Illumination light emitted from the light source device is incident on the light source connection portion 13, the illumination light is transmitted through the fiber probe 11, and is emitted in a radial direction (in all directions) from the light irradiation unit 12 with respect to a fiber probe center axis all.

The balloon catheter 20 includes a shaft 21, a balloon 22 and a multi-use port 23.

The shaft 21 is an elongated tube body having translucency and is made of nylon, silicon or Teflon (registered trademark).

The shaft 21 includes one lumen 24 extending along a shaft axis a21.

The lumen 24 of the shaft 21 is a lumen for injection. An inner diameter of the lumen 24 for injection is larger than an outer diameter of the fiber probe 11. Accordingly, the fiber probe 11 is insertable into the lumen 24 for injection of the shaft 21.

One opening of the lumen 24 for injection formed on one end side of the shaft 21 is an opening 24a for balloon. The balloon 22 is mounted on one end of the shaft 21 on a side of the opening 24a for balloon.

The balloon 22 is a bag which includes an opening at one end and has translucency. A balloon fixing portion 22a having an 0-ring shape is mounted on an opening side of the balloon 22.

The balloon fixing portion 22a is fixedly mounted at a preliminarily set position on an outer peripheral surface of a distal end side of the shaft 21 by welding or adhesion. The balloon 22 is inflated by supplying a liquid into the lumen 24 for injection and by injecting the liquid into the balloon 22 through the opening 24a for balloon.

The other opening of the lumen 24 for injection disposed on the other end side of the shaft 21 is an opening 24b for port. The multi-use port 23 is fixedly mounted on the other end portion of the shaft disposed on an opening 24b side for port. The multi-use port 23 includes two ports, that is, a port 25 for probe and a port 26 for liquid.

The port 25 for probe extends from a shaft proximal end portion in a proximal end direction along a shaft axis a21 which is a longitudinal axis of the shaft 21. The fiber probe 11 is inserted into the port 25 for probe through an insertion opening (not shown) on a proximal end surface side as indicated by an arrow. The fiber probe 11 passes through the port 25 for probe, and is inserted into the lumen 24 for injection of the shaft 21.

On the other hand, the port 26 for liquid extends obliquely from the side portion of the port 25 for probe in a proximal end direction in an intersecting manner with respect to the shaft axis a21 at a preliminarily set angle θ. The port 26 for liquid is a port for liquid injection, and for example, a syringe (not shown) is connected to the port 26 for liquid. As a result, a liquid in the syringe passes through the port 26 for liquid and the lumen 24 for injection, and is injected into the balloon 22 from the opening 24a for balloon.

Symbol 27a indicates a probe insertion section. A holding member (not shown) is disposed in the probe insertion section 27a. The holding member has a function of a sealing cock which prevents a liquid in the balloon 22 and the lumen 24 for injection from leaking to the outside, and a function of holding an arrangement position of the fiber probe 11 in the lumen 24 for injection.

Symbol 27b indicates a syringe mounting portion. The syringe can be detachably mounted on the syringe mounting portion 27b. The syringe is configured to be held in a state where the syringe is mounted on the syringe mounting portion 27b.

Accordingly, in a state where the balloon 22 of the balloon catheter 20 is inflated by injecting a liquid into the balloon 22, it is possible to prevent the liquid in the balloon catheter 20 from leaking to the outside and hence, the balloon 22 can be held in an inflated state.

The liquid discharge catheter 30 is an elongated tube body and is made of nylon, silicon or Teflon (registered trademark). The liquid discharge catheter 30 has one lumen 31 extending along a catheter axis a30.

The lumen 31 of the liquid discharge catheter 30 is a lumen for liquid discharge. An inner diameter of the lumen 31 for liquid discharge is set larger than an outer diameter of the balloon fixing portion 22a which is a maximum outer diameter within an effective length (L21 in the drawing) of the shaft 21 by a preliminarily set size. Accordingly, the shaft 21 provided with the balloon 22 can pass through the lumen 31 for liquid discharge of the liquid discharge catheter 30.

An opening 32 formed on one side of the lumen 31 for liquid discharge is disposed in a bladder. A balloon catheter insertion section (hereinafter, abbreviated as balloon insertion section) 34 is fixedly mounted on a side of another opening 33.

The shaft 21 provided with the balloon 22 of the balloon catheter 20 is insertable into the balloon insertion section 34. An end portion of the shaft 21 provided with the balloon 22 on a side of the opening 24a for balloon passes through the balloon insertion section 34, and the lumen 31 for liquid discharge, and is led to the outside of a distal end side of the liquid discharge catheter 30 from the opening 32 on one side.

More specifically, a balloon catheter effective length L20 is set greater than a liquid discharge catheter effective length L30 of the liquid discharge catheter 30 preliminarily. L30 indicates a distance from a distal end of the liquid discharge catheter 30 to a distal end surface of the balloon insertion section 34.

L21 indicates an effective length of the shaft 21 which is a distance from a distal end surface of the shaft 21 to a distal end surface of the port 25 for probe. L indicates a distance from a shaft distal end surface 21f to a balloon mounting portion 21a.

The balloon catheter effective length L20 is expressed by a following equation.

L20=L21−L

The balloon catheter effective length L20 is a value obtained by subtracting the distance L from the shaft effective length L21.

A urine discharge pack (not shown) is detachably mounted on the balloon insertion section 34.

A holding member (not shown) is mounted on the balloon insertion section 34. The holding member has a function of holding a state where the shaft 21 or the urine discharge pack is disposed in the balloon insertion section 34, and a function of a sealing cock which prevents a liquid in the lumen 31 from leaking to the outside in a state where the shaft 21 or the urine discharge pack is disposed in the balloon insertion section 34.

As described above, in the embodiment, the liquid discharge catheter 30 does not have two lumens, and has only one lumen 31 for liquid discharge. The shaft 21 on which the balloon 22 provided to the balloon catheter 20 is mounted on a side of the opening 24a for balloon is insertable into the lumen 31 for liquid discharge.

In addition to the above-mentioned configuration, the fiber probe 11 of the probe 10 for therapy is insertable into the lumen 24 for injection of the shaft 21.

Accordingly, it is possible to prevent the occurrence of a defect that insertability of the liquid discharge catheter 30 into a bladder is lowered by increasing an outer diameter of the liquid discharge catheter 30.

An optical therapeutic method using the optical therapeutic apparatus 1 is described hereinafter.

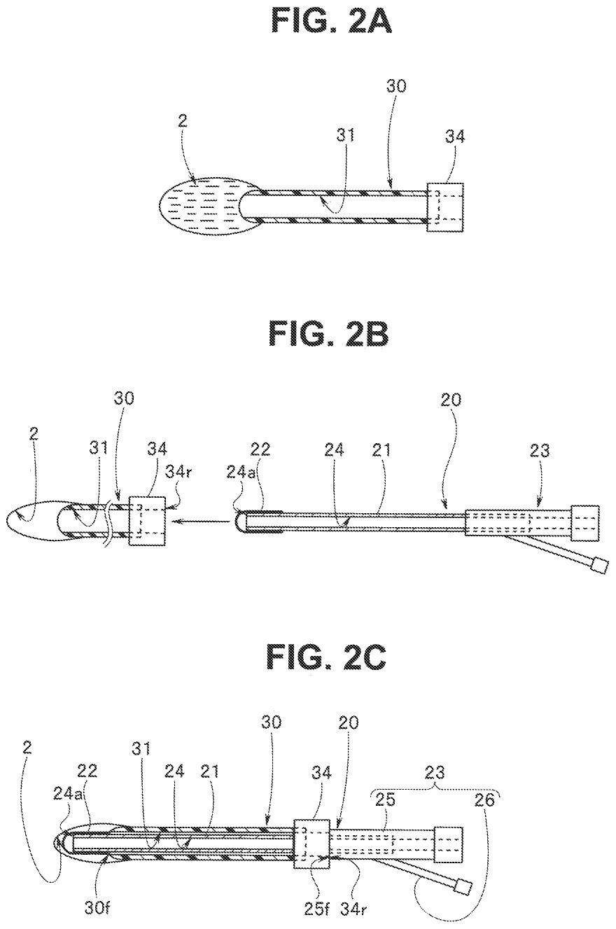

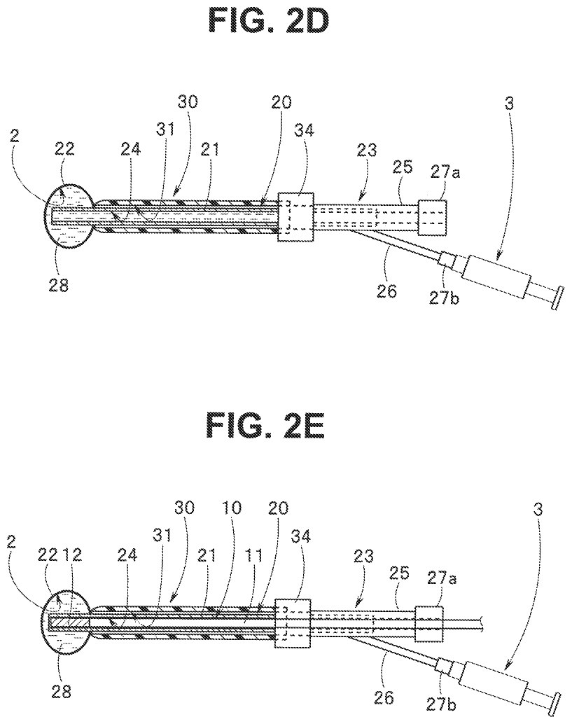

In starting an optical therapy of a bladder using the optical therapeutic apparatus 1, first, to discharge urine in the bladder to an outside, a medical staff member disposes the distal end portion of the liquid discharge catheter 30 having the lumen 31 for liquid discharge in the bladder 2 as shown in FIG. 2A.

Then, the medical staff member discharges the urine in the bladder to an outside through the lumen 31 for liquid discharge of the liquid discharge catheter 30.

Next, the medical staff member inserts the balloon catheter 20 into the liquid discharge catheter 30.

In other words, as shown in FIG. 2B, the medical staff member makes an end portion on a side of the opening 24a for balloon of the shaft 21 on which the balloon 22 is mounted face a proximal end surface 34r of the balloon insertion section 34. Then, the medical staff member inserts the end portion on the side of the opening 24a for balloon into the balloon insertion section 34, and guides the end portion into the lumen 31 for liquid discharge of the liquid discharge catheter 30.

Then, the medical staff member, as shown in FIG. 2C, brings the distal end surface 25f of the port 25 for probe which the balloon catheter 20 includes into contact with the proximal end surface 34r. As a result, a distal end portion of the shaft 21 is led out from the distal end surface 30f of the liquid discharge catheter 30, and the balloon 22 mounted on the shaft 21 is disposed in the bladder 2.

Next, the medical staff member mounts the syringe 3 in which, for example, distilled water is stored on the syringe mounting portion 27b of the port 26 for liquid. Then, injection of a liquid in the syringe 3 is started. Accordingly, a liquid 28 passes through the port 26 for liquid and the lumen 24 for injection, and is injected into the balloon 22. As a result, as shown in FIG. 2D, the balloon 22 is inflated along with the inflow of the liquid 28 and hence, the bladder 2 is brought into a state where the bladder 2 is inflated by the balloon 22.

Next, the medical staff member inserts a light irradiation unit 12 side of the fiber probe 11 of the probe 10 for therapy from the probe insertion section 27a of the port 25 for probe. Then, a light irradiation unit 12 side of the fiber probe 11 is inserted into the lumen 24 for injection and, as shown in FIG. 2E, a distal end of the fiber probe 11 is disposed at a preliminarily set position on a distal end side of the shaft 21. As a result, the light irradiation unit 12 is disposed in a vicinity of the opening 24a for balloon.

Then, light having a predetermined wavelength which is supplied from the light source device is emitted from the light irradiation unit 12 toward the bladder 2.

As described above, according to the embodiment, the light irradiation unit 12 of the fiber probe 11 is immersed in the liquid 28 injected into the balloon 22 for inflating the bladder 2, and is in a water-cooled state. Accordingly, when light is irradiated from the light irradiation unit 12, the generation of heat is attenuated by the injected liquid 28 and hence, breaking of the light irradiation unit 12 caused by heat can be prevented.

Since urine in the bladder is discharged preliminarily by the liquid discharge catheter 30, at the time of light irradiation, it is possible to prevent lowering of an amount of light caused by urine.

In the above-mentioned optical therapy, the balloon catheter 20 is inserted into the lumen 31 for liquid discharge of the liquid discharge catheter 30 and, thereafter, the fiber probe 11 is inserted into the lumen 24 for injection of the shaft 21.

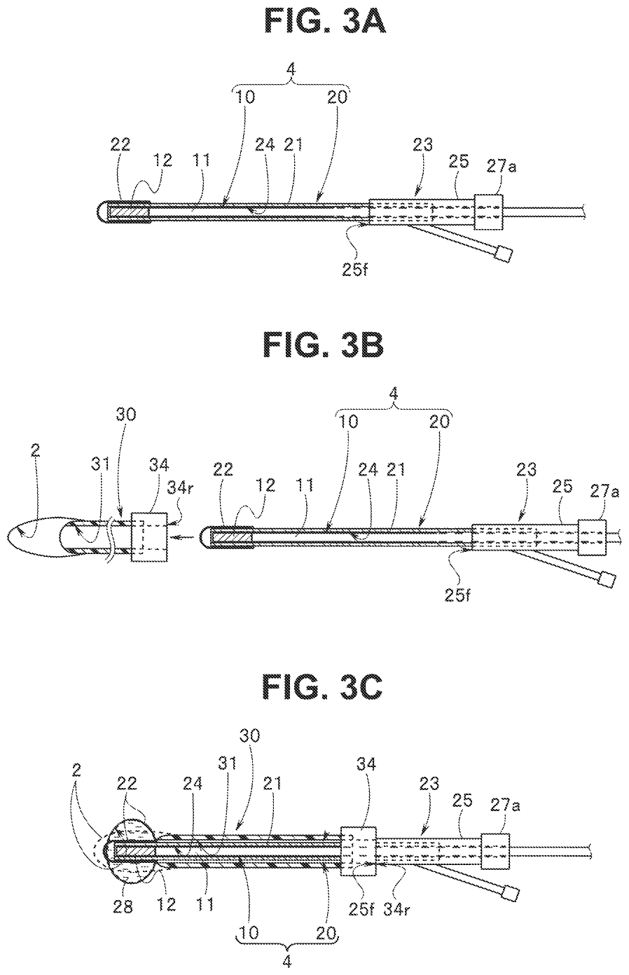

However, as shown in FIG. 3A, the probe 10 for therapy and the balloon catheter 20 may be integrally combined with each other and, thereafter, as shown in FIG. 3B, the probe 10 for therapy and the balloon catheter 20 may be inserted into the lumen 31 for liquid discharge of the liquid discharge catheter 30 after urine in the bladder is discharged.

In other words, before starting optical therapy of the bladder using the optical therapeutic apparatus 1, as shown in FIG. 3A, the medical staff member preliminarily disposes the light irradiation unit 12 of the fiber probe 11 of the probe 10 for therapy in the lumen 24 for injection of the shaft 21 of the balloon catheter 20. In other words, a probe-balloon integral set 4 which is obtained by integrally combining the probe 10 for therapy and the balloon catheter 20 is prepared.

Then, optical therapy of the bladder is started. First, as described above, a medical staff member disposes the distal end portion of the liquid discharge catheter 30 in the bladder 2 for discharging urine in the bladder to an outside in the same manner as described above, and discharges the urine in the bladder.

After the urine in the bladder is discharged, as shown in FIG. 3B, the medical staff member inserts the probe-balloon integral set 4 into the lumen 31 for liquid discharge of the liquid discharge catheter 30.

In this case, the medical staff member disposes the balloon 22 in the bladder 2 as indicated by a broken line in FIG. 3C. Then, the medical staff member inflates the bladder 2 while inflating the balloon 22 as indicated by a solid line by injecting the liquid 28 into the balloon 22 as described above.

In this manner, by preparing the probe-balloon integral set 4 in a preliminarily combined manner, after urine in the bladder 2 is discharged, therapy can be speedily performed by omitting a step of inserting the probe 10 for therapy.

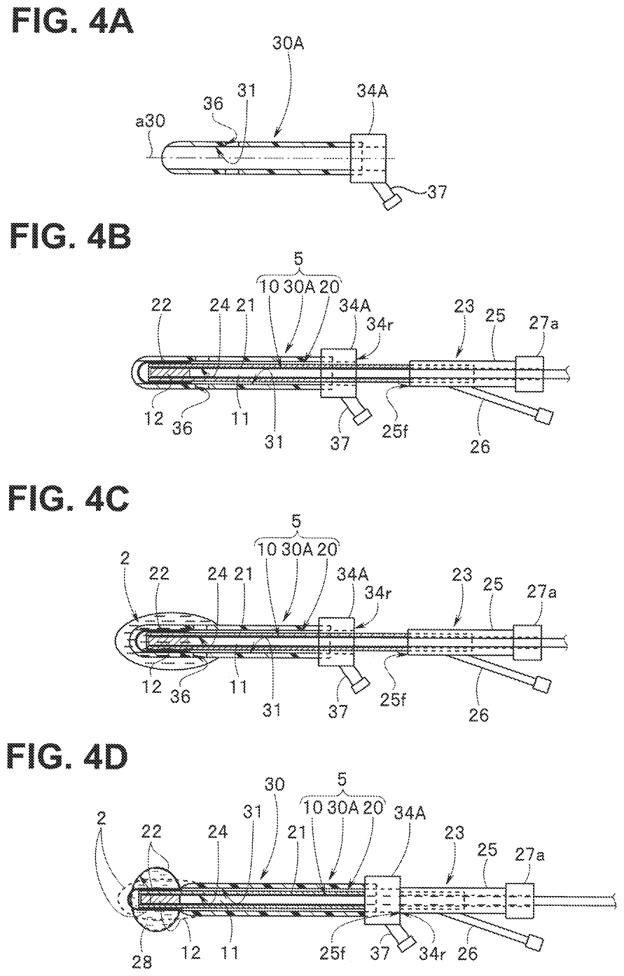

In the above-mentioned optical therapeutic apparatus 1, the liquid discharge catheter 30 includes one lumen 31 extending along the catheter axis a30. Further, the opening 32 on one side of the liquid discharge catheter 30 is disposed in the bladder, and the balloon insertion section is fixedly mounted on the other opening 33 side. However, as shown in FIG. 4A, a liquid discharge catheter 30A having the configuration shown in FIG. 4A may be adopted.

The liquid discharge catheter 30A shown in FIG. 4A includes: a lumen 31 for liquid discharge extending along a catheter axis a30; and a side hole 36 which has a center axis not shown which is intersect with the catheter axis a30. The side hole 36 is a through hole which makes the outside and the lumen 31 for liquid discharge communicate with each other. A plurality of, for example, two, side holes 36 are formed in the lumen 31 for liquid discharge in a circumferential direction.

Although the number of side holes 36 may be 1. However, the number of side holes 36 may preferably be plural, and the number of side holes 36 may be 3 or 4, for example.

In the embodiment, a balloon insertion section 34A is fixedly mounted on the other opening 33 side. A liquid discharge opening portion 37 is formed on a balloon insertion section 34A.

A shaft 21 provided with a balloon 22 of a balloon catheter 20 is insertable into the balloon insertion section 34A from a proximal end surface 34r side. A discharge urine pack (not shown) is detachably mounted on the liquid discharge opening portion 37.

The other components are substantially equal to the corresponding components of the above-mentioned embodiment and hence, the description of these components is omitted by adding the same symbols to the identical members.

An optical therapeutic method which uses the optical therapeutic apparatus 1 which includes a liquid discharge catheter 30A in place of the liquid discharge catheter 30 is described.

In the optical therapeutic apparatus 1 which includes the liquid discharge catheter 30A, before optical therapy is started, a medical staff member preliminarily prepares a probe-balloon-liquid discharge catheter integral set 5 shown in FIG. 4B which is an integral body formed of a probe 10 for therapy, the balloon catheter 20 and the liquid discharge catheter 30A.

The probe-balloon-liquid discharge catheter integral set 5 is formed by arranging a probe-balloon integral set 4 in the lumen 31 for liquid discharge of the liquid discharge catheter 30A. In such an arrangement state, a distal end of the probe-balloon integral set 4 is positioned at a position substantially equal to a position of the distal end of the liquid discharge catheter 30A or at a position in a slightly proximal end direction with respect to the position of the distal end of the liquid discharge catheter 30A.

In the embodiment, optical therapy is started by disposing the distal end portion of the liquid discharge catheter 30A of the probe-balloon-liquid discharge catheter integral set 5 in the bladder 2 as shown in FIG. 4C.

The probe-balloon-liquid discharge catheter integral set 5 is disposed in the bladder 2 filled with urine for discharging the urine in the bladder 2 and for performing therapy. Then, the urine in the bladder 2 mainly enters the lumen 31 for liquid discharge through the side holes 36, and is discharged from the liquid discharge opening portion 37.

After the urine in the bladder is discharged, the medical staff member withdraws the liquid discharge catheter 30A of the probe-balloon-liquid discharge catheter integral set 5 positioned in the bladder 2 and indicated by a broken line in FIG. 4D by a preliminarily set amount.

The proximal end surface 34r is brought into contact with a distal end surface 25f, and the balloon 22 mounted on the balloon catheter 20 is disposed in the bladder 2 in a state where the balloon 22 is exposed from a distal end surface of the liquid discharge catheter 30A.

At this stage of the operation, as described above, the bladder 2 is inflated while inflating the balloon 22 indicated by a solid line in FIG. 4D by injecting a liquid 28 in the balloon 22.

Other steps of the optical therapeutic method are substantially equal to the corresponding steps of the optical therapeutic method described previously.

By preparing the probe-balloon-liquid discharge catheter integral set 5 in a preliminarily combined form in this manner, optical therapy in the bladder can be performed more smoothly.

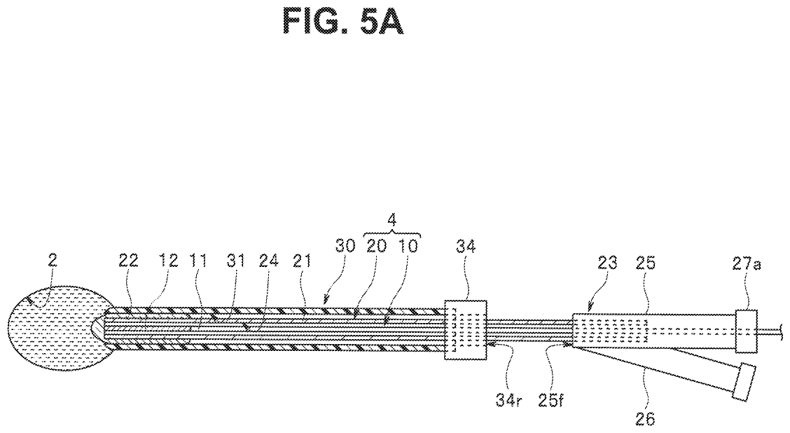

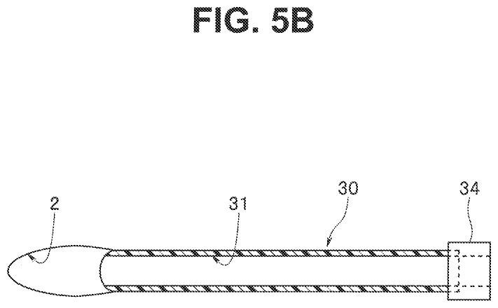

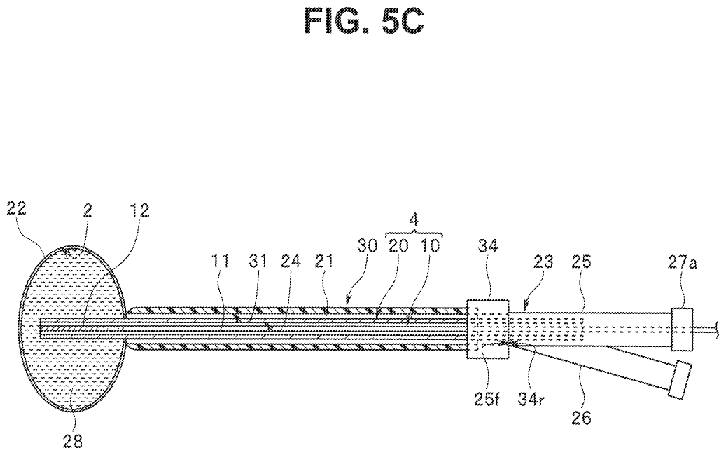

According to the embodiment, for example, optical therapy of a bladder may be performed in accordance with steps shown in FIG. 5A, FIG. 5B and FIG. 5C. The steps of such optical therapy are described hereinafter.

A medical staff member performs, for example, an operation of displacing the balloon catheter 20 while preventing the displacement of the probe 10 for therapy in a state where the probe-balloon integral set 4 is inserted into the lumen 31 for liquid discharge of the liquid discharge catheter 30. With such an operation, the balloon 22 is disposed at a predetermined position where the intrusion of a liquid from a distal end portion of the liquid discharge catheter 30 into the lumen 31 for liquid discharge is blocked.

In this step, the balloon 22 may be disposed at the predetermined position described previously. Accordingly, in this step, an operation of inserting the balloon catheter 20 into the lumen 31 for liquid discharge as a single body and displacing the balloon catheter 20 in the lumen 31 for liquid discharge may be performed in place of using the probe-balloon integral set 4.

The medical staff member inserts the probe-balloon integral set 4 into the lumen 31 for liquid discharge of the liquid discharge catheter 30, and disposes the distal end portion of the liquid discharge catheter 30 in the bladder 2 in a state where the balloon 22 is disposed at the predetermined position described previously, for example, such that the arrangement state is as shown in FIG. 5A.

After checking that the arrangement state is as shown in FIG. 5A, urine in the bladder 2 is discharged by removing the probe-balloon integral set 4 inserted into the lumen 31 for liquid discharge of the liquid discharge catheter 30, for example, as shown in FIG. 5B.

After checking that the urine in the bladder 2 is discharged as shown in FIG. 5B, the medical staff member disposes the balloon 22 in the bladder 2 by again inserting the probe-balloon integral set 4 into the lumen 31 for liquid discharge of the liquid discharge catheter 30 and bringing the distal end surface 25f into contact with the proximal end surface 34r.

For example, as shown in FIG. 5C, the medical staff member inflates the bladder 2 while inflating the balloon 22 disposed in the bladder 2 by injecting the liquid 28 into the balloon 22.

After checking that the balloon 22 and the bladder 2 are inflated as shown in FIG. 5C, the medical staff member performs an operation of emitting light having a predetermined wavelength from a light irradiation unit 12 disposed in the balloon 22 toward the bladder 2. In other words, with such an operation performed by the medical staff member, light having the predetermined wavelength which is supplied from a light source device is emitted from the light irradiation unit 12 disposed in the balloon 22 toward the bladder 2.

According to the steps described above, for example, even when a situation occurs where it is difficult to insert a distal end portion of the liquid discharge catheter 30 into the bladder 2, optical therapy can be performed by discharging urine in the bladder 2.

The present invention is not limited only to the embodiments described above, and various modifications can be carried out without departing from the gist of the present invention.

What is claimed is:

1. An optical therapeutic apparatus comprising:
a liquid discharge catheter including a lumen for liquid discharge, and a first tubular member with which a proximal end of the lumen for liquid discharge communicates;
a balloon catheter including a second tubular member provided with a shaft which includes one end and another end and is insertable into and removable from the lumen for liquid discharge of the liquid discharge catheter, a balloon mounted on the one end of the shaft, and a port disposed on the other end of the shaft and through which a liquid for inflating the balloon can be injected, the balloon catheter including a lumen for injection which makes the port and the balloon communicate with the shaft; and
a light irradiation body configured to emit light having a preliminarily set wavelength from a diffuse functional unit of a probe distal end in a state where the light irradiation body is inserted into the lumen for injection mounted on the shaft of the balloon catheter, the probe distal end is positioned in the balloon, and the liquid is injected into the balloon, wherein
the balloon is positioned to be exposed from a distal end of the liquid discharge catheter by bringing the second tubular member into contact with the first tubular member.

2. The optical therapeutic apparatus according to claim 1, wherein an effective length of the balloon catheter is greater than an effective length of the liquid discharge catheter.

3. The optical therapeutic apparatus according to claim 2, wherein the liquid discharge catheter includes a through hole through which the lumen for liquid discharge of the liquid discharge catheter and an outside communicate with each other, and the through hole intersects with a longitudinal axis of the liquid discharge catheter.

4. The optical therapeutic apparatus according to claim 1, wherein the port formed in the other end of the shaft includes a port for injection for injecting the liquid and a port for probe for allowing insertion and removal of the light irradiation body.

5. The optical therapeutic apparatus according to claim 4, wherein
the port for probe is disposed in an extending manner in a proximal end direction along a longitudinal axis of the shaft, and
the port for injection is disposed in an intersecting manner with the longitudinal axis at a preliminarily set angle.

6. The optical therapeutic apparatus according to claim 4, wherein a holding member which includes a function of a sealing cock which prevents the liquid injected into the lumen for injection of the balloon catheter from leaking to an outside is provided to the port for probe and the port for injection respectively.

7. The optical therapeutic apparatus according to claim 6, wherein the holding member provided to the port for probe holds a position of the light irradiation body disposed in the lumen for injection of the balloon catheter.

8. An optical therapeutic method comprising:
inserting a light irradiation body into a lumen for injection of a balloon catheter, the balloon catheter including a balloon on one end of a shaft, a port capable of injecting liquid for inflating the balloon on another end, and a lumen for injection communicating the port and the balloon with each other, and disposing a probe distal end of the light irradiation body in a vicinity of an opening for balloon of the lumen for injection;
introducing the shaft of the balloon catheter into a lumen for liquid discharge of the liquid discharge catheter, the lumen for liquid discharge being capable of discharging urine in a bladder;
disposing a distal end of the liquid discharge catheter in the bladder so as to dispose the balloon mounted on one end of the balloon catheter in the bladder;
removing the balloon catheter from the liquid discharge catheter and discharging the urine in the bladder;
inserting the balloon catheter into the lumen for liquid discharge and disposing the balloon in the bladder;
injecting the liquid into the balloon through the port of the balloon catheter and inflating the balloon by the liquid; and
emitting light having a preliminarily set wavelength from a light irradiation unit of the light irradiation body toward the bladder.
